(19) United States
(12) Patent Application Publication
(10) Pub. No.: US 2024/0325055 A1
(43) Pub. Date: Oct. 3, 2024

(54) FRICTION-FIT IMPLANTABLE DEVICES AND ASSEMBLIES

(71) Applicant: Orthofix US LLC, Lewisville, TX (US)

(72) Inventors: Frank LaRosa, Lewisville, TX (US); Nelson Li, Lewisville, TX (US); Kevin Roberts, Frisco, TX (US)

(21) Appl. No.: 18/620,291

(22) Filed: Mar. 28, 2024

Related U.S. Application Data
(60) Provisional application No. 63/493,058, filed on Mar. 30, 2023.

(51) Int. Cl. A61B 17/70; A61B 17/56

(52) U.S. Cl. CPC A61B 17/7037; A61B 17/7032; A61B 2017/564

(57) ABSTRACT

An implantable receiver may be configured to couple to a bone fastener for spinal fixation. The implantable receiver may include a friction-fit feature to allow the implantable receiver to maintain its orientation before being locked into place by a set screw. For example, the implantable receiver may have a body with an opening extending from the top to the bottom. The receiver may also have a pressure cap disposed within the opening of the body and a retainer ring disposed in a bottom portion the opening. The retainer ring may be expandable around a head of the bone fastener. The friction fit feature may be a compressible coil disposed around the opening of the body above the retainer ring. The compressible coil may be configured to contact the head of the bone fastener and deform to provide a frictional engagement between the head and the body.

### CROSS-REFERENCE TO RELATED APPLICATIONS

The present applications claims the benefit of, and priority to, U.S. Provisional Patent Application No. 63/493,058, filed Mar. 30, 2023, the entirety of which is incorporated by reference herein.

### TECHNICAL FIELD

This disclosure is generally directed to friction-fit devices and assemblies attachable to bone fasteners for implantation in an anatomy of a patient. For instance, one or more implantable assemblies including a receiver body coupled to a bone screw may be coupled to a connecting rod to retain one or more vertebrae in a desired relationship.

### BACKGROUND

Various systems for connecting fasteners (e.g., pedicle screws) to elongated supports (e.g., fixation rods) for the purposes of vertebral fixation have been proposed. Although described with reference to vertebral or spinal fixation, it should be appreciated that the systems described herein may be similarly applicable to other bone structures as well.

Generally, fixation systems include a receiver (or “receiver body” or “head”) which is attachable to both a fastener and a fixation rod to retain the rod in fixed relation to the fastener, and in turn, a vertebra into which the fastener is secured. Traditional receiver assemblies include a receiver and a fastener for attachment of fixation rod to a vertebra. A physician may use multiple receiver assemblies and/or multiple rods to secure the vertebrae in a desired spatial relationship. In some installations, a first rod may extend along a first side of a patient's spine and engage a first plurality of fastener assemblies each implanted in a different vertebra, and a second rod may extend along a second side of the patient's spine and engage a second plurality of fastener assemblies.

In some instances, a receiver assembly may come preassembled such that the receiver and fastener are preselected and attached to one another by the manufacturer. The assembly of the fastener and the receiver may involve special tools and trained technicians such that assembly by the physician, nurse, or surgical technician is impractical. Accordingly, the surgeon or technician may select a receiver and fastener assembly from a plurality of receiver and fastener assemblies based on the patient's anatomy and/or indications. Thus, the surgeon may be limited based on the variety of selections available at the time of surgery.

During a spinal fixation surgery, the receiver and fastener assemblies may be inserted through the patient's tissue via a surgical opening or ingress. The fasteners of each assembly may be driven into the patient's vertebra at desired locations. A connecting rod is then positioned through each receiver and the receivers and connecting rod are fixed in place by set screws or compression screws in each receiver. In order to position the connecting rod through each receiver, the receivers are oriented in alignment so that the connecting rod can be inserted through a channel or slot of each receiver. The alignment of the receivers can be a complicated part of the procedure. For example, gravity may cause the receivers to droop or slip out of alignment. Accordingly, the procedure may involve repositioning and/or reorienting one or more receivers multiple times before the connecting rod is successfully positioned through each receiver.

### SUMMARY

The present disclosure describes implantable devices and assemblies that provide a friction fit between a receiver and a fastener (e.g., bone screw). For example, a receiver may be configured to apply a frictional force to a screw head so that an orientation of the receiver can be maintained or stabilized relative to the screw head before the position is fixed by a set screw. Further, the implantable devices of the present disclosure may allow for modular assembly before or during a spinal fixation procedure. For example, the implantable device may allow for bottom-side loading of the screw into the receiver so that various screws having various characteristics (e.g., length, diameter, etc.) can be coupled to the receiver body.

One embodiment of the present disclosure includes an implantable receiver configured to couple to a head of a bone fastener. The implantable receiver may have a body, a pressure cap and a compressible coil. The body may have a top, a bottom, and an opening extending from the top to the bottom. The pressure cap may be disposed within the opening of the body. The retainer ring disposed in a bottom portion of the body within the opening, wherein the retainer ring may be expandable around a head of the bone fastener. The compressible coil may be disposed around the opening of the body above the retainer ring. The compressible coil may be configured to contact the head of the bone fastener and deform to provide a frictional engagement between the head of the bone fastener and the body.

In some embodiments, the compressible coil may be toroidally shaped. The compressible coil may have a cross-sectional surface that is circularly shaped in one embodiment and is elliptically shaped in another embodiment. Moreover, in some embodiments, the cross-sectional surface of the compressible coil may be angled outward and in other embodiments may be angled inward. The compressible coil may comprise a compressible polymer. However, the compressible coil may also be coiled coil. The compressible coil may comprise one or more of stainless steel, titanium, an alloy comprising cobalt and chromium, poly carbonate urethane (PCU), or polyether ether ketone (PEEK). The compressible coil may be disposed in a coil recess. In some instances, the body may also comprise a pin that is receivable within a slot on the pressure cap.

The present disclosure also includes a fastener assembly which may comprise a receiver and a screw. The receiver may comprise a body having an opening, a pressure cap disposed within the opening of the body, a retainer ring disposed within the opening proximate to a bottom of the opening, and a compressible coil disposed in the opening above the retainer ring and below the pressure cap. The bone fastener may have a head insertable through the bottom of the opening. The retainer ring may be expandable around the head and the compressible coil may be configured to deform to apply frictional force to the head.

In some embodiments, the compressible coil is toroidally shaped. The compressible coil may have a cross-sectional surface that is circularly shaped in some embodiments and is elliptically shaped in other embodiments. In some embodiments, the compressible coil may comprise a helically-wound or spiral-shaped wire, filament, strand, or other elongate structure. The compressible coil may be disposed in a coil recess defined in the body.

The present disclosure also includes a method of assembling an implantable device. The method may comprise the steps of: providing a body comprising a top, bottom, and an opening extending from the top to the bottom; positioning a retainer ring in a recess of the opening proximate to the bottom of the body; positioning a compressible coil in a recess of the opening above the retainer ring; coupling a pressure cap within the opening above the compressible coil; and, inserting a bone fastener into the bottom of the opening; wherein the retainer ring expands around a head of the bone fastener; and, wherein the compressible coil deforms to apply a friction force to the head.

In some embodiments, the compressible coil may be toroidally shaped. In some embodiments, the step of coupling a pressure cap may also include affixing a pin to the body such that an end of the pin is received in a slot of the pressure cap.

These and other objects, features and advantages of this invention will become apparent from the following detailed description of the various aspects and principles of the invention taken in conjunction with the accompanying drawings.

### BRIEF DESCRIPTION OF THE DRAWINGS

The accompanying drawings, which are incorporated in and constitute a part of the specification, illustrate embodiments of the invention and, together with the detailed description herein, serve to explain the principles of the invention. The drawings are only for purposes of illustrating examples and are not to be construed as limiting the invention. It is emphasized that, in accordance with the standard practice in the industry, various features are not drawn to scale. In fact, the dimensions of the various features may be arbitrarily increased or reduced for clarity of discussion.

FIG. 1 is a perspective view of a spinal fixation system including friction-fit pedicle screw assemblies and a connecting rod in accordance with an embodiment of the present disclosure.

FIG. 2A is a perspective view of a receiver for a friction-fit pedicle screw assembly in accordance with an embodiment of the present disclosure.

FIG. 2B is an exploded view of the receiver in FIG. 2A in accordance with an embodiment of the present disclosure.

FIG. 3A is a side perspective view of the receiver in FIG. 2A with a transparent body.

FIG. 3B is a bottom perspective view of the receiver in FIG. 2A with a transparent body.

FIG. 4A is a cross-sectional view of a receiver for a friction-fit pedicle screw assembly in accordance with an embodiment of the present disclosure.

FIG. 4B is a cross-sectional view of a pedicle screw assembly, including the receiver shown in FIG. 4A and a screw, in accordance with an embodiment of the present disclosure.

FIG. 4C is a cross-sectional view of a pedicle screw assembly, including the receiver shown in FIG. 4A and a screw, in accordance with an embodiment of the present disclosure.

FIG. 4D is a cross-sectional view of a pedicle screw assembly, including the receiver shown in FIG. 4A and a screw, in accordance with an embodiment of the present disclosure.

FIG. 5A is a cross-sectional view of a portion of a coil for a friction-fit pedicle screw assembly in accordance with an embodiment of the present disclosure.

FIG. 5B is a cross-sectional view of a portion of a coil for a friction-fit pedicle screw assembly in accordance with an embodiment of the present disclosure.

FIG. 5C is a cross-sectional view of a portion of a coil for a friction-fit pedicle screw assembly in accordance with an embodiment of the present disclosure.

FIG. 5D is a cross-sectional view of a portion of a coil for a friction-fit pedicle screw assembly in accordance with an embodiment of the present disclosure.

FIG. 5E is a cross-sectional view of a portion of a coil for a friction-fit pedicle screw assembly in accordance with an embodiment of the present disclosure.

FIG. 6A is a front view of a screw for use in a friction-fit pedicle screw assembly in accordance with an embodiment of the present disclosure.

FIG. 6B is a front view of a screw for use in a friction-fit pedicle screw assembly in accordance with an embodiment of the present disclosure.

FIG. 6C is a front view of a screw for use in a friction-fit pedicle screw assembly in accordance with an embodiment of the present disclosure.

FIG. 7 is a flow chart illustrating a method of implanting a friction-fit pedicle screw assembly in accordance with an embodiment of the present disclosure.

FIG. 8 is a cross-sectional view of a receiver for a friction-fit pedicle screw assembly in accordance with an embodiment of the present disclosure.

### DETAILED DESCRIPTION

For the purpose of promoting an understanding of the principles of the present disclosure, reference will now be made to the implementations illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In addition, this disclosure describes some elements or features in detail with respect to one or more implementations or figures, when those same elements or features appear in subsequent figures, without such a high level of detail. It is fully contemplated that the features, components, and/or steps described with respect to one or more implementations or figures may be combined with the features, components, and/or steps described with respect to other implementations or figures of the present disclosure. For simplicity, in some instances the same or similar reference numbers are used throughout the drawings to refer to the same or like parts.

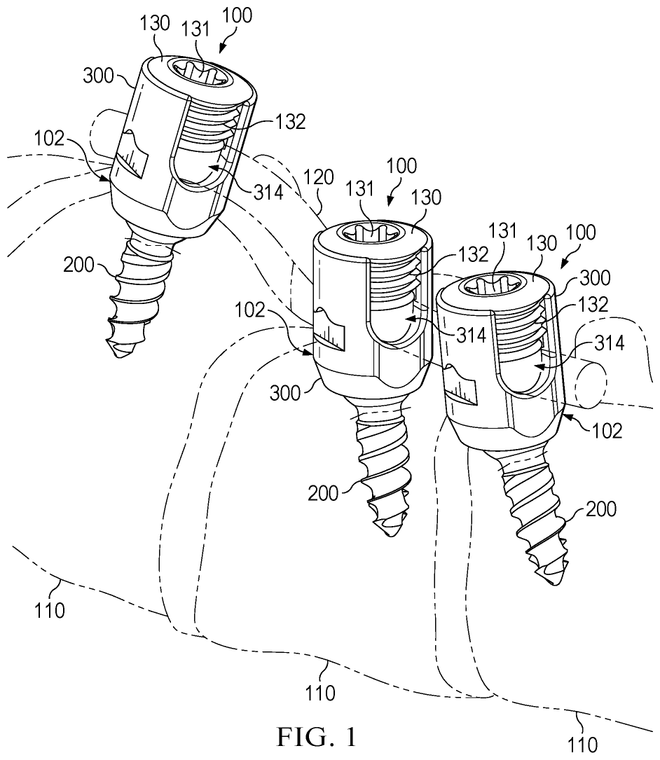

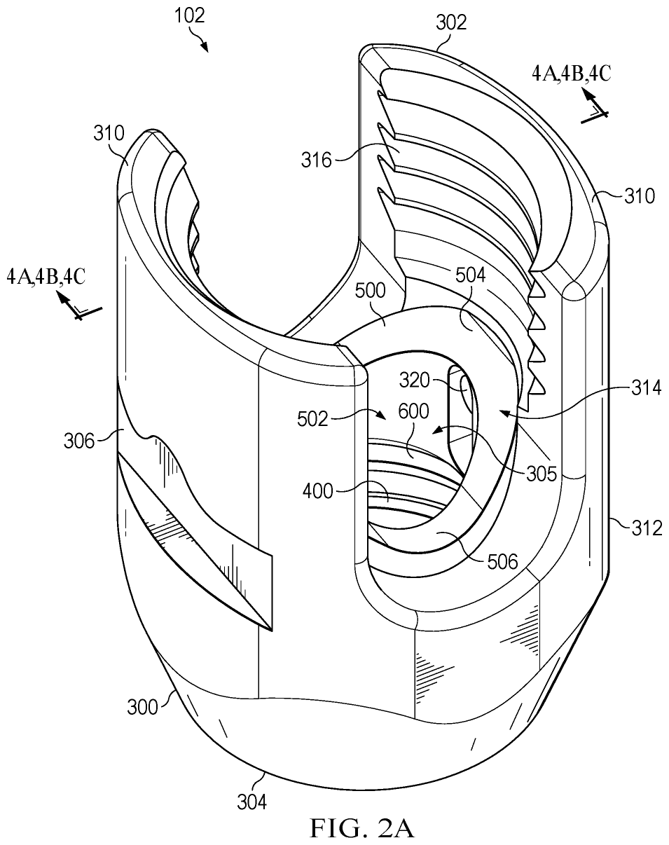

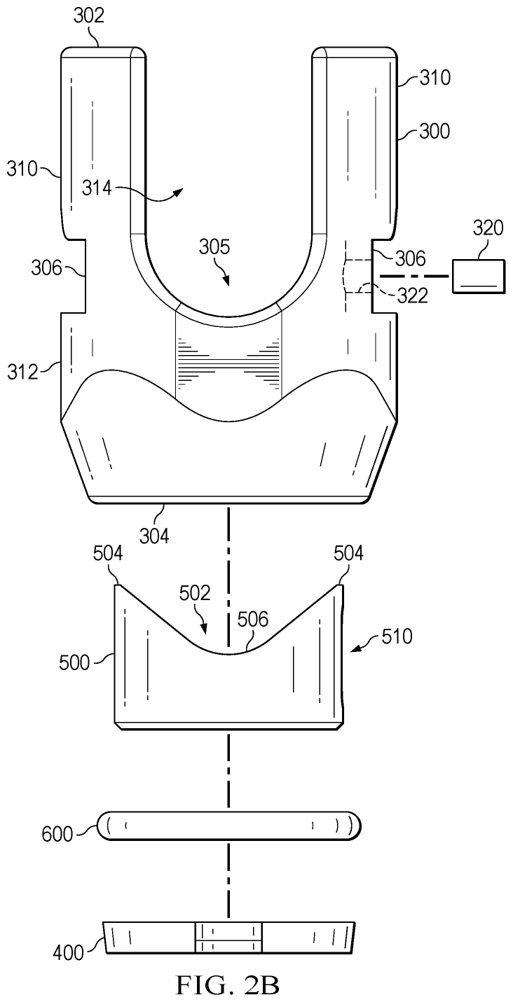

FIG. 1 is a perspective view of a plurality of pedicle screw assemblies 100 including a plurality of implantable receivers 102 coupled to respective vertebrae 110 of a patient's spine by a plurality of screws 200. Each implantable receiver 102 in FIG. 1 includes a body 300, and may further include a retainer ring (400, FIG. 2B), a pressure cap (500, FIG. 2B), and a coil (600, FIG. 2B) as described in more detail below. The receivers 102 are coupled to one another by a rod 120 positioned in U-shaped slots 314 or saddles of the receivers 102. The rod 120 may be sized, shaped (e.g., bent, curved), and otherwise structurally configured to correct a spinal deformity, and/or to retain the vertebrae 110 in a fixed position. The positions and orientations of the receivers 102 relative to the rod 120 and the bone screws 200 may be fixed or otherwise retained by the set screws 130. For example, the bone screws 200 may be coupled to the receivers 102 in a multi-axial relationship such that the bone screws 200 may be rotated about at least one axis relative to the respective receiver 102. For example, in some aspects, one or more of the bone screws 200 may include spherical, semi-spherical, or otherwise round screw heads (not shown) seated within the receiver 102. The receivers 102 may be configured to rotate, tilt, swivel, twist, and/or otherwise move relative to the screw heads of the bone screws 200. With the bone screws 200 fixed to the vertebrae 110, a physician may move the receivers 102 into the orientation shown in FIG. 1 to receive the rod 120. The orientations of the receivers 102 relative to one another may be maintained by the friction-fit coupling to the bone screws 200 as the physician guides the rod 120 through the slots 314. With the rod 120 in the slots 314, and with the desired overhang of the rod 120 from the outermost receivers 102, the set screws 130 can be tightened down to compress rod 120 and the screw heads of the screws 200 against the base of the receivers 102 to fix the position and orientation of the receivers 102 relative to the rod 120 and bone screws 200. The set screws 130 may be any appropriate shape. For example, the set screws 130 may include a drive feature 131 that receives an instrument for tightening the set screw 130 to compress the rod 120 and the screw heads of the screws 200. The drive feature 131 may be hexalobe shaped as shown in the illustrated embodiment, or may be any other appropriate shape including hexagonal, square, or triangular. Additionally, the set screws 130 may have threading 132 along part or all of the length. The threading 132 of the set screw 130 may threadably engage the receivers 102.

FIG. 2A is a perspective view of a receiver 102 according to an embodiment of the present disclosure. FIG. 2B shows an exploded view of the receiver 102 shown in FIG. 2A. The embodiment of the receiver 102 shown in FIG. 2A may be similar or identical to the receivers 102 shown in FIG. 1, in some aspects. The receiver 102 includes a body 300, a retainer ring 400 or lock ring, a pressure cap 500, and a coil 600. The body 300 has a top end 302 for receiving a set screw (e.g., 130, FIG. 1) and a rod (e.g., 120, FIG. 1) and a bottom end 304 for receiving a screw head. The body 300 may further comprise an opening 305. The opening 305 may pass from the top end 302 to the bottom end 304. In the illustrated embodiment, the body 300 is tulip-shaped, meaning the body 300 has two arms 310 on either side of the body 300 that extend from a base 312 of the body 300 to the top 302. The arms 310 define a channel or U-shaped slot 314 for seating a rod. The arms 310 may be referred to as sidewalls, wings, or any other suitable term. The body 300 is configured to receive a connecting rod via the U-shaped slot 314. Moreover, the body 300 further includes internal threads 316 on the interior surfaces of the arms 310. The threads 316 may be configured to engage corresponding threads on a set screw (e.g., 130, FIG. 1). The set screw may be tightened down into the body 300 to compress the connecting rod onto the pressure cap 500. Compressing the pressure cap 500 may also cause the pressure cap 500 to put additional pressure onto the screw head 210 of the screw 200 to fix the receiver 102 in a desired position and orientation.

The body 300 also has two engagement features 306 that may provide for releasable engagement with a tool for inserting, positioning, and/or removing the receiver 102. For example, the engagement features 306 may provide for releasable engagement with a tool for inserting the subassembly including the receiver 102 and the connected screw 200, and driving the screw 200 into the patient's bone (e.g., vertebra). In the illustrated embodiment, the engagement feature 306 is centered with the arm 310. It will be understood that the other arm 310 may also include an engagement feature similar or identical to the engagement feature 306. The engagement feature 306 on the other arm 310 may also be centered on the arm 310. The centering of the engagement feature 306 may be beneficial for robust engagement with the insertion tool. For example, the centered placement of the engagement feature 306 may allow for a deeper groove or impression of the engagement feature 306 into the arm 310. In another aspect, the top end 302 of the body 300 may be associated with a frangible portion or breaking line of the body 300. For example, in some embodiments, the body 300 may be integrally formed with extension portions or tower portions extending proximally from the top end 302. The area of the body 300 comprising the top end 302 may comprise a weakened portion.

The receiver 102 may further comprise a pin 320 that is received in a pin hole 322 in the side of the body 300. The pin 320 projects into the opening 305 of the body 300. The pin 320 may be welded, adhered, soldered, threadably attached, and/or otherwise affixed, attached, or coupled to the body 300. In other embodiments, the pin 320 may be formed in the body 300. The pressure cap 500 has a slot 510 that is shaped to receive the pin 320. This allows the pressure cap 500 to move up and down along the opening 305 of the body 300, but minimizes the rotation of the pressure cap 500 so that it remains in a relatively constant orientation.

The receiver 102 also includes a pressure cap 500, which may also be referred to as a pressure member. The pressure cap 500 includes a concave upper surface or top surface for receiving the connecting rod, as described above. The pressure cap 500 may be saddle-shaped, meaning the pressure cap 500 has two ends 504 with an arched surface forming a depression 506 between the two ends 504. This saddle-shape may generally match the shape of the U-shaped slot 314 formed between the arms 310 of the body 300. Thus, the pressure cap 500 may be shaped to accept a rod that is placed within the U-shaped slot 314 of the body 300. The pressure cap 500 may also include a concave surface on the bottom side of the pressure cap 500 to contact and engage a top surface of a screw head. However, in other embodiments, the pressure cap 500 may have any appropriate shape having a top for seating a rod and a bottom for contacting a screw head. For example, the pressure cap 500 may include a v-shaped depression, a rectangular depression, an elliptical depression, a hexagonal depression, and/or any other suitable shape for receiving the connecting rod. Similarly, the bottom surface of the pressure cap 500 may be flat, inclined, saddle-shaped and may be shaped elliptically, rectangularly, hexagonally or any other suitable shape for contacting and engaging a top surface of a screw head.

Moreover, the pressure cap 500 has an opening 502 extending through the center and aligning with the opening 305 of the body 300. The opening 502 allows an instrument to access a head of a screw when it is inserted into the receiver 102. For example, an interfacing portion or bit of a screw driver may be able to pass through the opening 502 of the pressure cap 500 so that the bone screw may be screwed into bone.

The retainer ring 400 and coil 600 are located within a base 312 of the body 300 and will be described in more detail below.

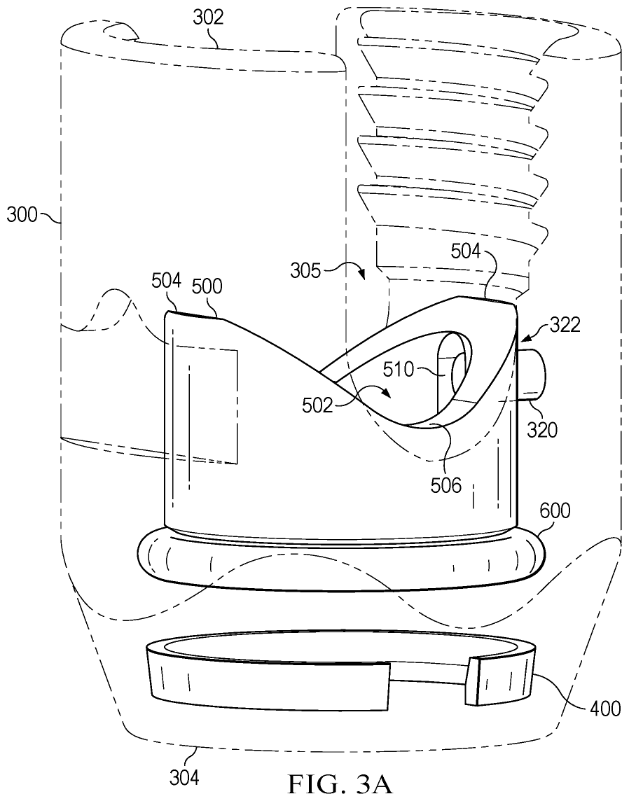

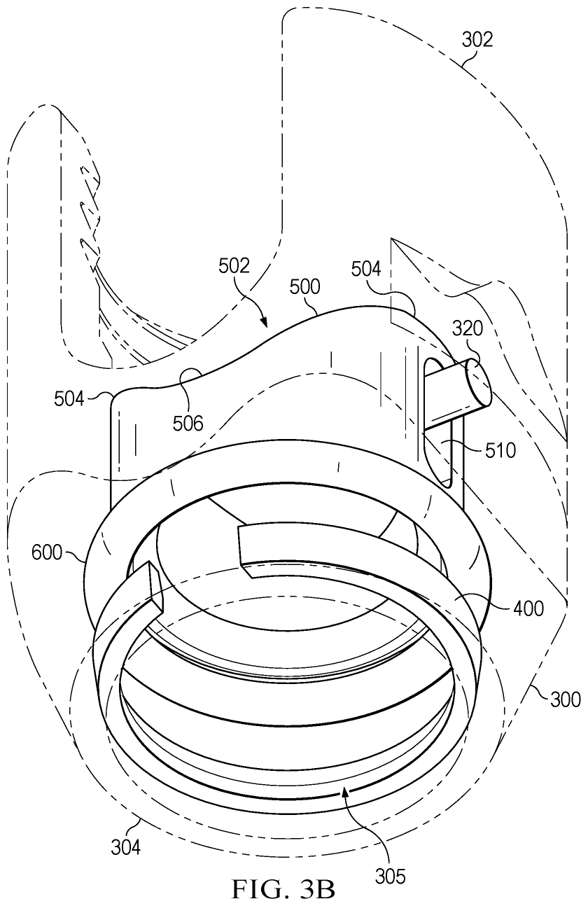

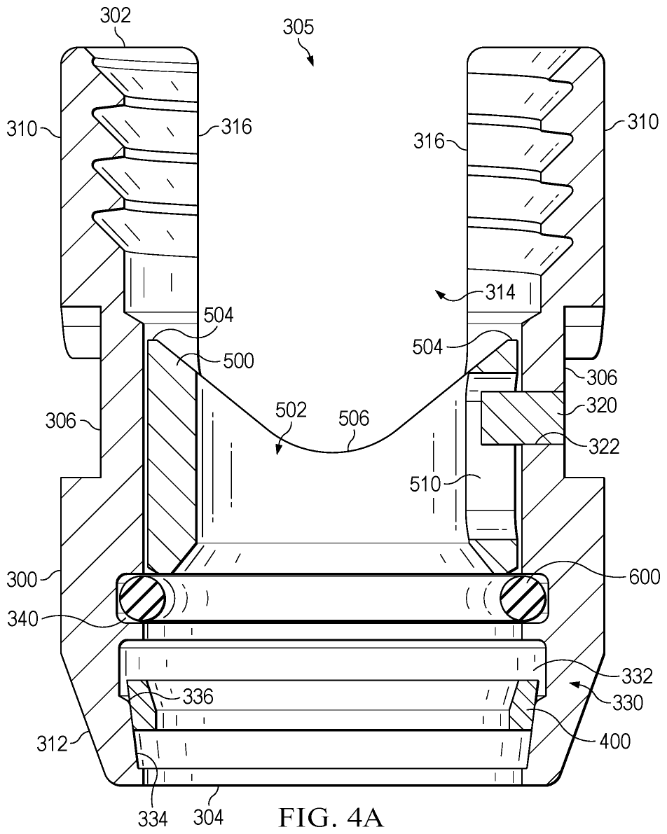

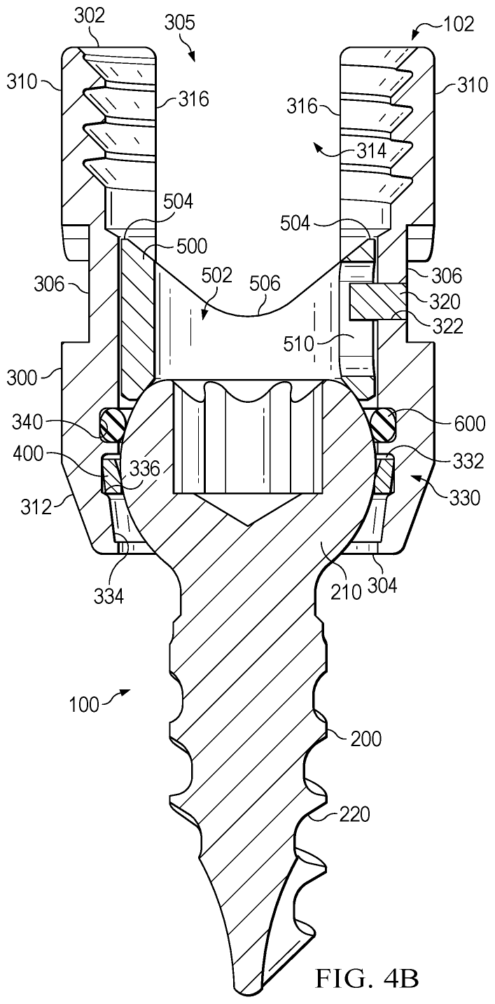

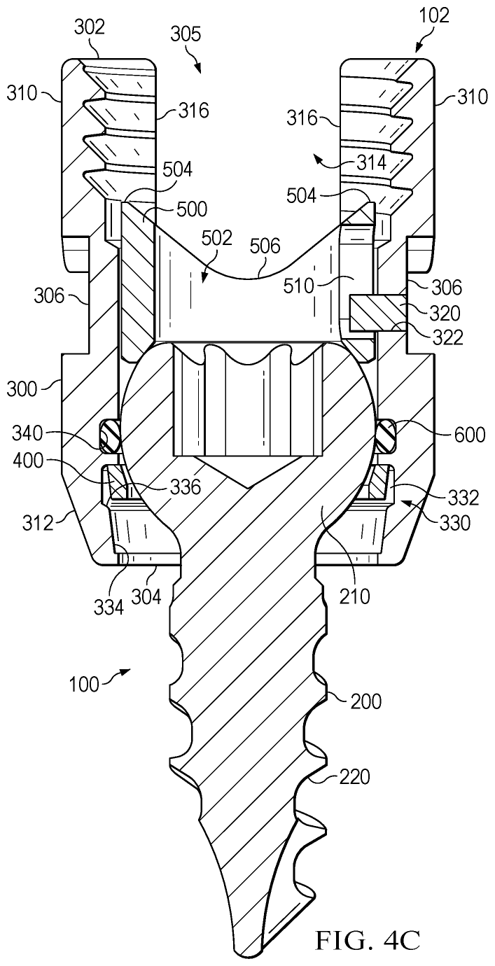

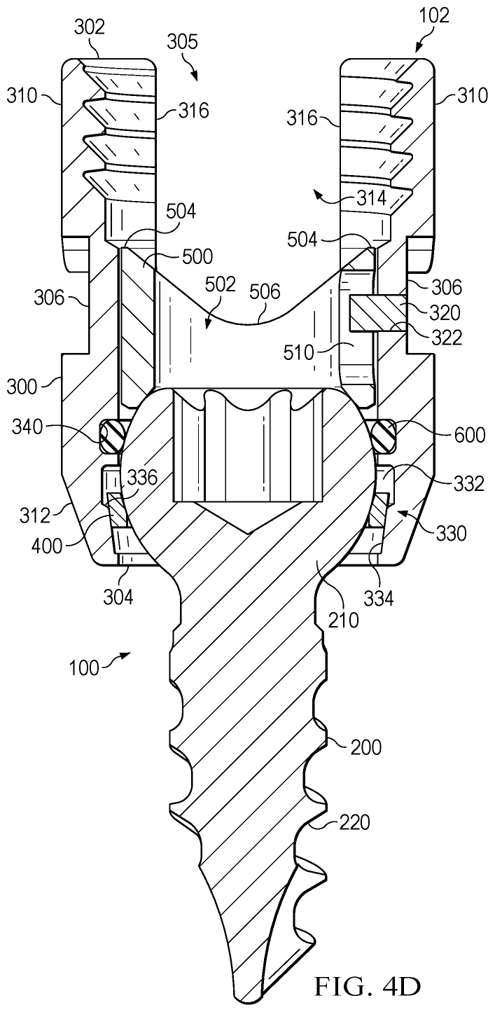

FIGS. 3A and 3B show perspective views of the receiver 102 with the body 300 shown as being transparent for illustrative purposes. FIG. 3A shows a perspective side view of the receiver 102 and FIG. 3B shows a perspective bottom view of the receiver 102. When the receiver 102 is assembled, the retainer ring 400 is located around the opening 305 proximate the bottom 304 of the body 300. In this embodiment, the retainer ring 400 is a split ring that has a discontinuous annular shape configured to expand and/or retract to enlarge and/or reduce an inner diameter of the retainer ring 400. In other embodiments, the retainer ring 400 may be a continuous ring capable of expanding over a screw head when it is inserted from the bottom 304 of the body 300. The retainer ring 400 may be configured to lock the screw 200 into the retainer 102 once the screw head 210 has been inserted through a bottom opening of the retainer ring 400, as shown in FIGS. 4B and 4C, for example. In some embodiments, the upper surface of the screw head 210 may be spherical, rounded, tapered, or otherwise configured to cause the retainer ring 400 to expand as the screw head 210 is pressed against the retainer ring 400 to allow the screw head 210 to pass through the retainer ring 400. Once the screw head 210 has passed through the retainer ring 400, the retainer ring 400 may relax and contract to lock against a bottom curved surface of the screw head 200. In some embodiments, an inner surface of the retainer ring 400 includes a ridge or seating feature configured to engage the bottom surface of the screw head 210. However, in other embodiments, the receiver 102 may have any appropriate component that locks the screw head 210 into the receiver 102, such as spring-loaded ball bearings, yielding locking ridge, and/or any other suitable feature.

In the illustrated embodiment, the coil 600 is located around the opening 305 of the body 300 above the retainer ring 400 and below the pressure cap 500. The coil 600 is toroidally-shaped in this embodiment. However, in other embodiments, the coil 600 may have another shape. For example, the coil 600 may not be a complete ring as shown, but may instead have one or more gaps. Moreover, the coil 600 may be elliptically-shaped or hexagonally-shaped, for example. In FIGS. 1-5, the coil 600 is shown as being solid. However, as discussed in more detail below, a coil according to the present disclosure need not be a solid and may instead be a coil 600. In the illustrated embodiment, the coil 600 is a continuous loop. Even though the coil 600 is shown as including a circular loop, the loop may be in any other appropriate shape including an ellipse, a rectangle, or a hexagon. In other embodiments, the coil 600 may also comprise one or more disconnected sections along the opening 305 of the body 300. The one or more discontinuous sections of coil 600 may be linear, curved, or angled.

Moreover, in other embodiments, the coil 600 may be located below the retainer ring 400 or may be located on an inner surface of the retainer ring 400. In yet other embodiments, there may be more than one coil 600 and the coils may all be located between the pressure cap 500 and the retainer ring 400, all be located below the retainer ring 400, all be located on an inner surface of the retainer ring, or may be located in any combination of locations.

The coil 600 is compressible so that when a screw head is inserted through the bottom 304 of the body 300, the coil 600 deforms around the screw head. In this way, the coil 600 applies a frictional force to the screw head. The frictional force (e.g., static friction) induced by the coil 600 may be sufficient to retain the position and orientation of the receiver 102 relative to the screw head 210. For example, the friction may be sufficient to overcome gravitational forces acting on the receiver 102 to keep the receiver 102 from drooping, sagging, or sliding after the physician has positioned the receiver 102 in alignment to receive a rod.

As illustrated in FIGS. 4A, 4B, and 4C, the receiver 102 may allow for bottom-loading of the screws 200 through the bottom 304 of the opening 305 of the body 300. FIG. 4A shows a cross-sectional view of the receiver 102 through the arms 310 (according to the line shown in FIG. 2A). FIGS. 4B and 4C show the same cross-sectional view as FIG. 4A, but of the assembly of the pedicle screw assembly 100 (the receiver 102 with a screw 200 inserted). In some embodiments, the receiver 102 may be configured for assembly before or during a surgical procedure. For example, the physician may select the screw 200 based on the patient's anatomy and indications. In some embodiments, the screw 200 may be selected after the surgery has begun and after the surgeon has created an access through the patient's tissue to the bone. In other instances, the physician and/or surgeon may select the screw 200 before the surgery based on medical images of the patient's anatomy (e.g., x-ray, computed tomography, magnetic resonance imaging).

In some aspects, a physician may load the screw 200 into the receiver 102 to form a pedicle screw assembly 100 prior to inserting and driving the screws 200 into the patient's bone. The bottom-loading style of the assembly may be referred to as a modular assembly. The bottom-loaded modular assembly may be advantageous, in some aspects. For example, the modular assembly style of the receivers 102 may allow for the physician to choose a type and/or size of screw and assemble the receiver 102 and screw 200 during a spinal fixation procedure, based on the patient's anatomy and indications. The modular style may also allow for quick and efficient assembly with little or no disassembly of the receiver 102.

The upper surface of the screw head 210 may include a spherical, aspherical, or otherwise curved shape configured to engage the bottom surface of the pressure cap 500. In other embodiments, the screw head 210 may include a conic section shape. Accordingly, the screw head 210 may be curved about at least one axis to allow the screw head 210 to continuously rotate relative to the pressure cap 500. In other embodiments, the screw head 210 may include a polygonal shape having a plurality of flat surfaces arranged around an axis of the screw 200. For example, the screw head 210 may include, on the upper surface, 10, 20, 25, 30, or any other suitable number of flat surfaces arranged around the axis of the screw 200. The number of flat surfaces on the upper surface of the screw head 210 may correspond to the number of possible orientations of the receiver 102 about the longitudinal axis of the screw 200. In some embodiments, the pressure cap 500 may include corresponding polygonal surfaces on the bottom side or surface of the pressure cap 500.

The screw 200 includes a distal threaded shaft 220 comprising screw threads configured to drive into and engage the patient's bone. In the illustrated embodiment, the threads are right-handed threads. In other embodiments, the threads may be left-handed threads. The threads may have any suitable pitch, depth, and/or other geometric characteristics based on the target bone or tissue and application for the assembly. The screw 200 may be machined, laser sintered, 3D printed, or otherwise manufactured by any suitable manufacturing process. It will be understood that the threaded portion of the shaft of the screw 200 may extend a greater or lesser portion of the shaft than what is shown in FIGS. 4B and 4C.

In this embodiment, the retainer ring 400 is disposed within a retainer ring recess 330 within the base 312 of the body 300. The retainer ring recess 330 allows the retainer ring 400 enough room to expand over the screw head 210. The retainer ring recess 330 is wider at the top than at the bottom. Thus, as the screw head 210 is pushed upward, the retainer ring 400 moves up and expands outward, allowing the screw head 210 to pass through the retainer ring 400. In the illustrated embodiment, the retainer ring recess 330 has an upper portion 332 and a lower portion 334. The upper portion 332 is wider than the lower portion 334 such that there is a shoulder 336 or ledge between the upper portion 332 and the lower portion 334. The lower portion 334 is provided and shaped to allow sufficient room for the screw head 210 to pass through, and the retainer ring 400 can expand in upper portion 332, as explained further below. The retainer ring 400 may be initially disposed at the region of the ledge or shoulder 336 between the upper 332 and lower 334 portions or in the upper portion 332 before the screw 200 is inserted. The retainer ring 400 may then be pressed upward into the upper portion 332 as the screw head 210 is inserted through the bottom of the body 300 of the receiver 102. As the retainer ring 400 moves into the upper portion 332, the retainer ring 400 may expand more than it expands in the lower portion 334. When the screw head 210 is fully seated, the retainer ring 400 may be disposed between the upper 332 and lower 334 portions or in the upper portion 332. In some embodiments, when the screw head 210 is fully seated, the retainer ring 400 rests on the shoulder 336 in the retainer ring recess 330. In some embodiments, when the screw head 210 is fully seated, the retainer ring 400 may not be disposed in the lower portion 334. The upper portion 332 and/or lower portion 334 may be tapered so that the upper portions 332 and/or lower portion 334 are wider at the top than at the bottom. However, in other embodiments, may have a different shape. In some embodiments, the retainer ring recess 300 does not have distinct upper 332 and lower 334 portions, but tapers from the top to the bottom. In some embodiments, the retainer ring recess 330 may not allow the retainer ring 400 to move up and down and may only allow the retainer ring 400 to expand outward. In other embodiments, the body 300 may not have a retainer ring recess 330 and instead may be attached to the wall of the opening 305 through another appropriate method.

Moreover, the coil 600 is disposed within a coil recess 340 within the base 312 of the body 300. The coil recess 340 is shaped to hold the coil 600 while allowing it to deform. In the illustrated embodiment, the coil recess 340 is a distinct recess from the retainer ring recess 330 such that material of the body 300 separates the coil recess 340 from the retainer ring recess 330. The material between the coil recess 340 and the retainer ring recess 330 may form a ridge or a shelf between the two recesses. When the screw head 210 is pushed up through the retainer ring 400, the screw head 210 deforms the coil 600 as shown in FIG. 4B. However, in other embodiments, the coil recess 340 may be another appropriate shape. For example, the coil recess 340 may be larger than the coil recess 340 shown, allowing the coil to move within the coil recess 340. In other embodiments, the body 300 may not have a coil recess 340; instead, the coil 600 may be attached to the wall of the opening 305 itself (for example, via an adhesive) or may be integrally formed with the opening 305. In yet other embodiments, there may be a single recess in the body 300 that seats both the coil 600 and the retainer ring 400 instead of including a separate coil recess 340 and retainer ring recess 330.

The receiver 102 may be assembled by placing the coil 600 in the coil recess 340. The coil 600 may sit in the coil recess 340 or may be affixed to the coil recess 340 using, for example, an adhesive. The coil 600 may be inserted through either the top 302 or bottom 304 of the opening 305. The retainer ring 400 may be placed in the retainer ring recess 330. The retainer ring 400 may sit in the retainer ring recess 330 or may be affixed to the retainer ring recess 330 using, for example, an adhesive. The retainer ring 400 may be inserted through either the top 302 or bottom 304 of the opening 305. Moreover, the pressure cap 500 may be inserted into the opening 305 such that the slot 510 aligns with the pin hole 322. The pin 320 may then be inserted through the pin hole 322 and the slot 510. The pin 320 may be affixed to the pin hole 322 by any appropriate means including by, for example, a mechanical lock or an adhesive. The receiver 102 may be assembled in any order. For example, the receiver 102 may be assembled in the following order: coil 600, retainer ring 400, pressure cap 500, pin 320. In another example, the receiver 102 may be assembled in the following order: pressure cap 500, pin 320, coil 600, retainer ring 400.

The pedicle screw assembly 100 may be assembled by inserting the screw 200 through the bottom 304 of the receiver 102 until it is locked within the receiver 102. FIGS. 4B-4D show the pedicle screw assembly 100 as it is being assembled. FIG. 4B shows a cross section of the pedicle screw assembly 100 as the screw 200 is being pushed upwards through the bottom 304 of the receiver 102. As shown in FIG. 4A, before the screw head 210 is inserted, the retainer ring 400 may be disposed between the upper 332 and lower 334 portions of the retainer ring recess 330. Thus, as the screw head 210 is pushed upwards, the retainer ring 400 is pushed upward into the wider upper portion 332 of the retainer ring recess 330 and expands around the screw head 210. While the screw head 210 is pushed upwards, the screw head 210 also contacts the coil 600 and deforms the coil 600 outwards. When the coil 600 is in this deformed configuration, the coil 600 applies a frictional force to the screw head 210. Moreover, the top of the screw head 210 contacts the bottom of the pressure cap 500 as it is pushed upwards, moving the pressure cap 500 upwards. As the pressure cap 500 moves upwards, the slot 510 moves along the pin 320, thus maintaining the same orientation of the pressure cap 500 as it moves. The pin 320 may contact the bottom of the slot 510, preventing the pressure cap 500 and thus the screw 200 from moving further upwards. Once the screw head 210 is locked into the receiver 102, the frictional force applied by the coil 600 will hold the receiver 102 in the same position relative to the screw 200. However, a surgeon may move or pivot the receiver 102 to a different position as desired.

FIG. 4C shows the pedicle screw assembly 100 when the screw head 210 is pushed up to the furthest point within the body 300 of the receiver 102. As described above, the retainer ring recess 330 may be wider at the top than at the bottom, giving the retainer ring 400 additional room to expand when the retainer ring 400 is pushed to the top of the retainer ring recess 330 so that the screw head 210 can pass through the expanded retainer ring 400. Once the retainer ring 400 has passed over the widest part of the screw head 210, the retainer ring 400 may contract, locking the screw head 210 into the receiver 102, as shown in FIG. 4C. In some embodiments, the retainer ring 400 may contract to a width slightly larger than the width of the lower portion 334 of the retainer ring recess 330. The pressure cap 500 may be disposed at a maximum position within the body 300 such that the pin 320 contacts the bottom of the slot 510 within the pressure cap 500. The pin 320 may prevent the screw head 210 from moving further up into the body 300 by preventing the pressure cap 500 from moving further up within the body. The screw head 210 may continue to contact and deform the coil 600. In some embodiments, the widest part of the screw head 210 may contact the coil 600 and deform the coil 600 to the maximum tension, at which the coil 600 exerts the most frictional force on the screw head 210. In other embodiments, the coil 600 may contact another part of the screw head 210 such that it is not deformed to maximum tension.

FIG. 4D shows the pedicle screw assembly 100 after assembly. After the screw 200 is pressed upward through the bottom 304 of the receiver 102, the upward pressure on the screw 200 can be removed, thus allowing the screw 200 to settle into an assembled configuration. The screw 200 may be move downward slightly within the receiver 102 such that the bottom of the screw head 210 rests within the base 312 of the body 300. In some embodiments, as the retainer ring 400 contracts, the retainer ring 400 may move downward in the retainer ring recess 330. The screw head 210 may also press downward on the retainer ring 400 as it moves downward within the receiver 102. When the screw head 210 is seated within the receiver 102, the retainer ring 400 may be disposed between the upper 332 and lower 334 portions of the retainer ring recess 330. In some embodiments, the retainer ring 400 may rest on the shoulder 336 of the retainer ring recess 330 such that it is disposed within the upper portion 332. Moreover, as the screw 200 moves downward, the pressure cap 500 may move downward as well such that the bottom of the pressure cap 500 maintains contact with the top of the screw head 210. The slot 510 of the pressure cap 500 may slide downward such that the pin 320 is located at the middle or top of the slot 510. In some embodiments, as the screw 200 moves downward, the coil 600 remains in the same location. The coil 600 remains in contact with the screw head 200 as the screw moves downward. Although the coil 600 may still be deformed, it may expand slightly. However, in other embodiments, the coil 600 may not expand or may deform further when a downward force on the screw 200 is removed. In some embodiments, the coil 600 may move downwards as the upward force on the screw 200 is removed. When the screw head 210 is fully seated within the receiver 102, the coil 600 applies a frictional force to the screw head 210.

In other embodiments, the screw 200 may not move downward when the upward force is removed. Accordingly, the pressure cap 500 may not move downward when the upward force is removed from the screw 200. In some embodiments, the retainer ring 400 may not move downward when the upward force is removed from the screw 200. Instead, the retainer ring 400 may remain in the same location and may contract. In other embodiments, the retainer ring 400 may not contract and may remain expanded. In yet other embodiments, the retainer ring 400 may move downward in the retainer ring recess 330 even if the screw 200 does not move downward.

The materials of the receiver 102 may be biocompatible, and may have other structural characteristics appropriate for use in spinal fixation. For example, the body 300, pressure cap 500, pin 320, retainer ring 400, and/or the screw 200 may include a biocompatible metal, such as stainless steel, titanium, and/or alloys thereof. In other embodiments, one or more components of the receiver 102 may include a polymer material, such as DELRIN, polyether ether ketone (PEEK), polytetrafluoroethylene (PTFE), polysulfone (PS), polycarbonate, and/or any other suitable polymeric material. One or more components of the receiver 102 may be manufactured by milling, machining, casting, molding, laser sintering, 3D printing, and/or any other suitable process. The components of the receiver 102 may be formed of the same materials or of different materials.

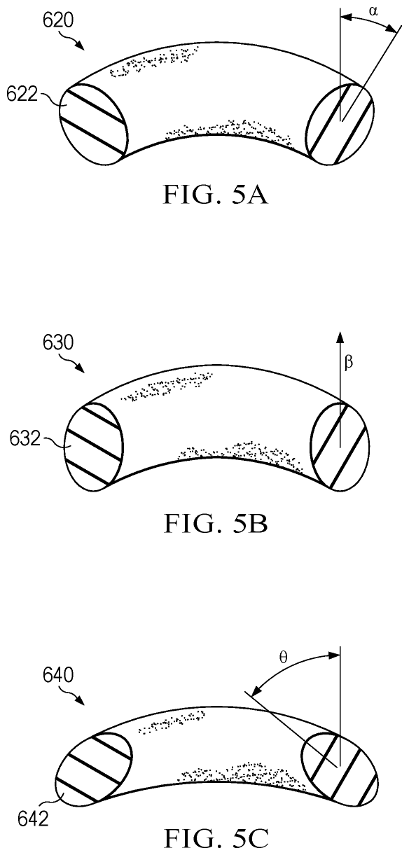

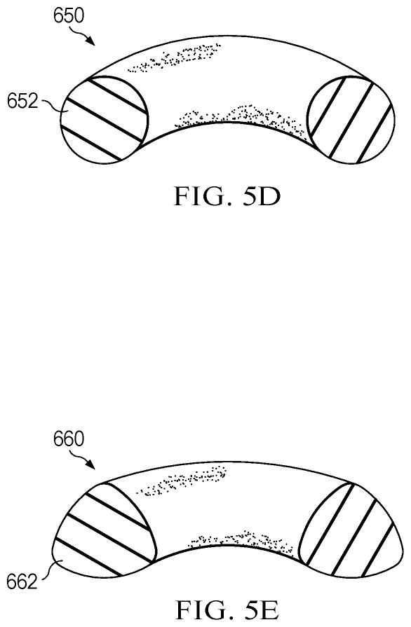

The coil 600 may be formed in a variety of shapes and materials. FIGS. 5A-5E show portions of different embodiments of a coil according to the present disclosure. In some embodiments, the coil 600 may be a canted coil having an oval cross-section. For example, FIGS. 5A-5C each show a canted coil 620, 630, 640 with an oval-shaped cross-sectional face 622, 632, 642. Moreover, the oval-shaped face 622, 632, 642 may be angled relative to a vertical axis. In FIG. 5A, the coil 620 has an oval-shaped face 622 that is angled outward from the center of the coil 620 as indicated by angle α. Angle α may be any angle ranging from 0° to 90°. In FIG. 5B, the coil 630 has an oval-shaped face 632 that is aligned with the vertical axis. In other words, the angle β is 0°. In FIG. 5C, the coil 640 has an oval-shaped face 642 that is angled inward towards the center of the coil 640 as indicated by angle θ. Angle θ may be any angle ranging from 0° to 90°.

However, a coil need not have an oval-shaped cross-sectional face and instead may be any other appropriate shape. For example, in one embodiment shown in FIG. 5D, the coil 650 has a circularly-shaped face 652. In another embodiment shown in FIG. 5E, the coil 660 may have a triangularly-shaped face 662. In other embodiments, a cross-sectional face of the coil may be square-shaped, hexagonally-shaped, or another shape and may be at any angle.

In FIGS. 5A-5B, the coil is shown as being solid or hollow. However, as discussed earlier, the coil may be a coiled in some embodiments. A solid or hollow coil may be comprised of any appropriate compressible, biocompatible material. If the coil 600 is coiled, it may be comprised of any appropriate biocompatible material such that the coil itself is compressible. For example, the coiled coil 600 may be comprised of a metal like titanium, stainless steel, an alloy comprising cobalt and chromium, poly carbonate urethane (PCU), polyether ether ketone (PEEK), and other implantable grade plastics.

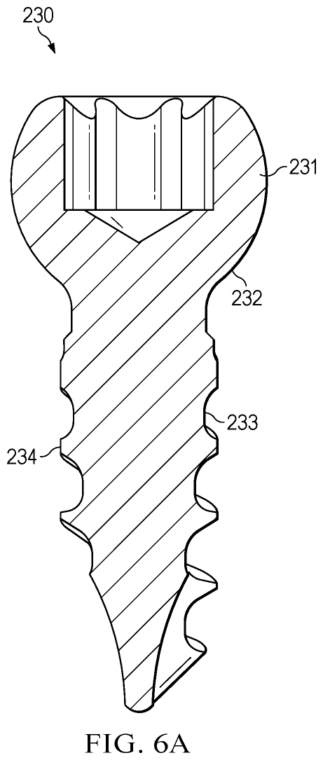

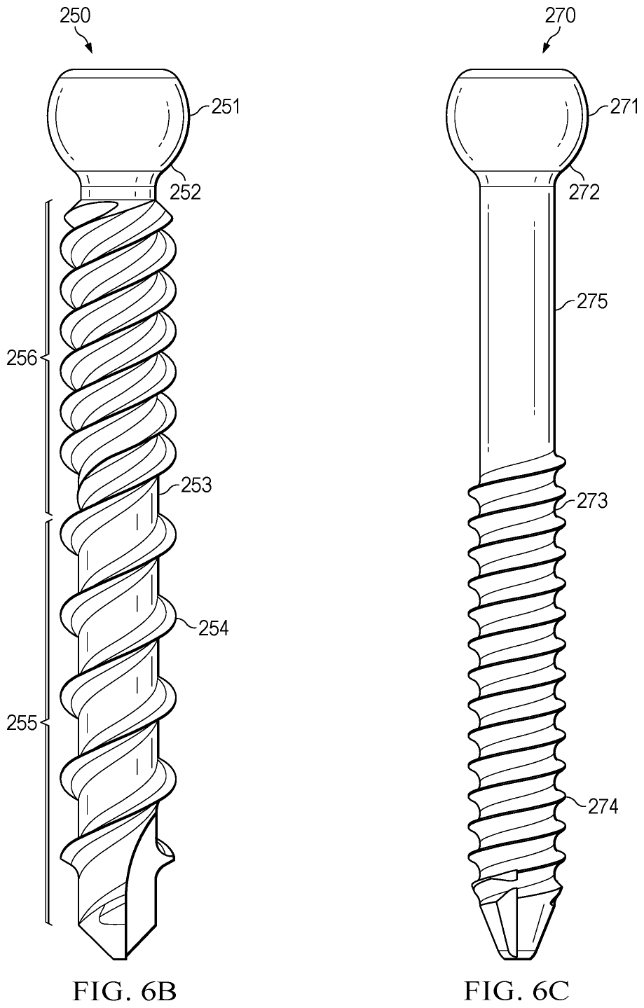

FIGS. 6A-6C show various embodiments of a screw 200 for use in the pedicle screw assembly 100. FIG. 6A illustrates a dual lead pitch screw 230 as shown in FIGS. 1, 4B, and 4C. In the illustrated embodiment, the dual lead pitch screw 230 comprises a head 231 having a spherical-shaped bottom 232 and a shaft 233. The shaft 233 comprises threading 234 that extends along the length of the shaft 233. The threading 234 of the dual lead pitch screw comprises two starts. The dual lead pitch screw 230 may be advantageous for use in cortical bone. However, in other embodiments, the screw 250 may not be dual lead and instead may have one start or more than two starts. Moreover, the screw 250 may have any appropriate pitch or lead.

FIG. 6B illustrates a part dual lead, part single lead screw 250. This embodiment comprises a head 251 having a spherical-shaped bottom 252 and a shaft 253. The shaft 253 comprises a threading 254 that extends completely or partially down the length of the shaft 253. Unlike the embodiment illustrated in FIG. 6A, the threading 254 in the embodiment illustrated in FIG. 6B comprises a first portion 255 that has one start and a second portion 256 that has two starts. The first portion 255 and the second portion 256 may be any appropriate length. This embodiment may be advantageous for use in bone that comprises a cortical layer and a cancellous layer. In other embodiments, the threading may comprise any appropriate number of portions with any appropriate number of starts. Moreover, the screw 250 may have any appropriate pitch or lead.

FIG. 6C illustrates a smooth shank screw 270. This embodiment comprises a head 271 having a spherical-shaped bottom 272 and a shaft 273. In this embodiment, the shaft 273 comprises threading 274. The threading 274 in screw 270 illustrated in FIG. 6C has threading 274 that extends partially along the length of the shaft 273. Thus in the embodiment of FIG. 6C, the screw 270 comprises a smooth portion 275 between the threading 274 and the head 271. The length of the smooth portion 275 may be any appropriate length. Moreover, the screw 270 may comprise any appropriate number of starts and may comprise multiple portions with different numbers of starts.

Any of the screws described herein may be any appropriate length. For example, the screws may be 20 mm, 25 mm, 30 mm, 35 mm, 40 mm, 45 mm, 50 mm, 55 mm, 60 mm, or any other length.

The receiver 102 of the pedicle screw system 100 may be compatible with any of the screws 230, 250, 270 shown in FIGS. 6A, 6B, 6C, respectively, or any other appropriate screw according to the embodiments contemplated by the present disclosure. Thus, the same receiver 102 may be used for any appropriate screw 200. This allows the physician to have one type of receiver 102 but choose the desired screw 200 during the procedure. In other aspects, the receiver 102 may be specifically sized, shaped, or otherwise configured for use for one type of screw 200 but not for a different type of screw.

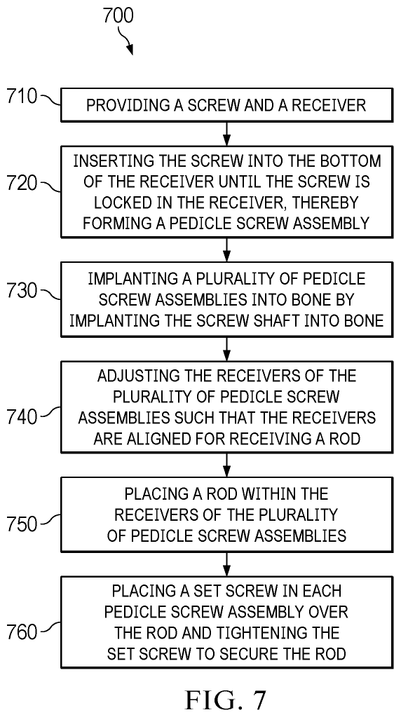

FIG. 7 shows a method 700 of assembling treating a spinal condition using the pedicle screw assembly 100. Step 710 of the method 700 includes providing a screw 200 and a receiver 102. The receiver 102 may be any receiver 102 described herein, including any of the receivers 102 or parts of receivers 102 shown in FIGS. 1-5E. The screw 200 may be any screw 200 described herein, including the screws 200 shown in FIG. 1, 4B, or 4C. The surgeon or technician may select a receiver and fastener assembly from a plurality of receiver and fastener assemblies based on the patient's anatomy and/or indications. Step 720 of the method includes inserting the screw 200 into the bottom 304 of the receiver 102 until the screw 200 is locked in the receiver 102, thereby forming a pedicle screw assembly 100. As the screw 200 moves upward through the receiver 102, the retainer ring 400 may expand over the head 210 of the screw, the pressure cap 500 may be pressed upward, and the coil 600 may be deformed as described in reference to FIG. 4B. When the upward force is removed from the screw 200, the screw 200 may move downward slightly to rest in the base 312 of the body 300. As the screw 200 moves downward, the retainer ring 400 may contract and move downward, the pressure cap 500 may move downward and the coil 600 may remain in the same location, but may expand slightly as described in reference to FIG. 4C. However, the screw 200 may be inserted into and be locked in the receiver 102 in any way as described herein.

Step 730 of the method 700 includes implanting a plurality of pedicle screw assemblies 100 into bone by implanting the screw shaft 220 into the bone. In some embodiments, the bone may be a vertebrae 110. As described herein, an instrument may releasably engage the engagement feature 306 of the receiver 102. The instrument may then be used to position the pedicle screw assembly 100 at the desired position. The same instrument or a different instrument may drive the screw 200 into the bone. For example, a screw driver may pass through the opening 502 of the pressure cap 500 to access the screw head 210. The screw driver may then be used to screw the screw shaft 220 into the bone. However, the screw 200 may be driven into the bone using any appropriate method. Step 740 of the method 700 includes adjusting the receivers 102 of the plurality of pedicle screws 100 such that the receivers 102 are aligned for receiving a rod 120. An instrument may engage with the engagement feature 306 of the receiver 102 to move the receiver 102 independent of the screw 200. The receiver 102 may be moved into any appropriate position for receiving a rod. The frictional force that the coil 600 applies to the screw head 210 may retain the position and orientation of the receiver 102 relative to the screw head 210.

Step 750 of the method 700 includes placing a rod 120 within the receivers 102 of the plurality of pedicle screw assemblies 100. Once the receivers 102 are aligned, a rod 120 may be placed such that it fits within the U-shaped slot 314 formed by the arms 310 of the receiver 102. The rod 120 may be bent or curved into the desired shape before or while placing the rod 120 into the receiver 102. Step 760 of the method 700 includes placing a set screw 130 in each pedicle screw assembly 100 over the rod 120 and tightening the set screws 130 to secure the rod 120. Tightening the set screws 130 may also secure the position and orientation of the receivers 102 relative to the screws 200. The set screws 130 may be any appropriate set screw 130 design, including the design shown in FIG. 1. The set screw 130 may have threads that engage the threads 316 of the arms 310 of the body 300. An instrument, like, for example, a screw driver, may be used to tighten the set screw 130 until it contacts and presses against the rod 120. The set screws 130 may secure the rod 120 and/or the receivers such that they do not move. In some embodiments, the rod 120 may be tightened such that it stabilizes the vertebrae 110.

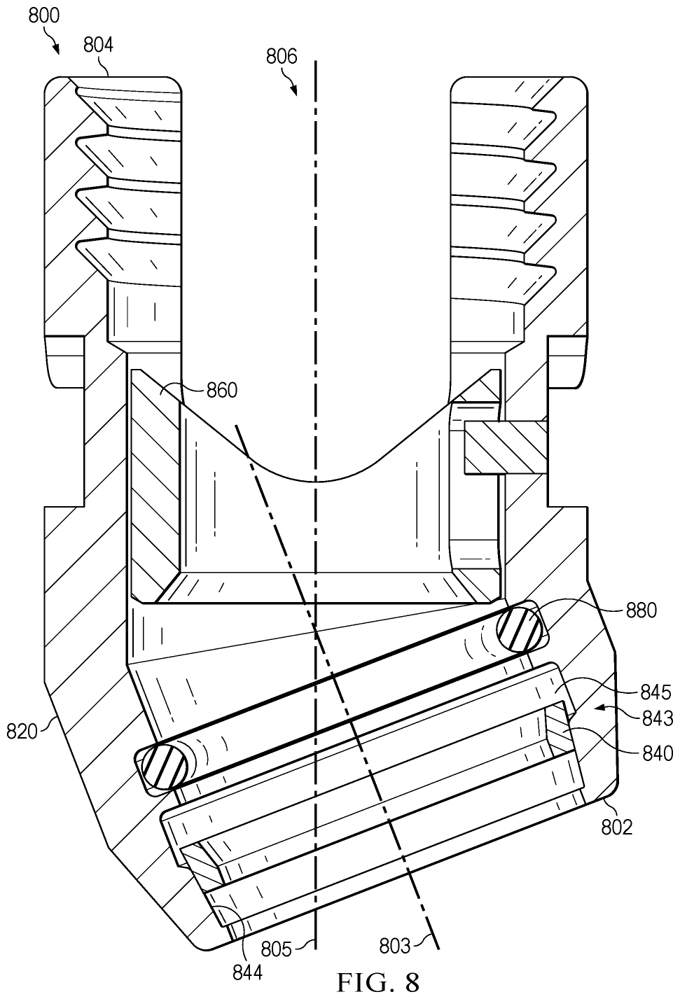

FIG. 8 illustrates another embodiment of a receiver 800, according to some aspects of the present disclosure. Receiver 800 is similar to receiver 102 illustrated in FIGS. 1-4D in that the receiver 800 comprises a body 820, a retainer ring 840, a pressure cap 860, and a coil 880. However, in receiver 800 shown in FIG. 8, the bottom 802 of the opening 806 is angled with respect to the top 804 of the opening. In other words, a first axis 803 passing through the center of the bottom 802 of the opening 806 is angled with respect to a second axis 805 passing through the center of the top 804 of the opening 806. The pressure cap 840 is generally the same as pressure cap 500.

The retainer ring recess 843 comprises a lower portion 844 and an upper portion 845 wider than the lower portion 844. Before the screw head 210 is inserted, the retainer ring 840 may be disposed between the upper 845 and lower 844 portions. When the screw head 210 is pressed upwards into the opening 806 of the receiver 800, the retainer ring 840 may be pressed upward into the wider upper portion 845 and expand around the screw head 210. Once the retainer ring 840 passes over the widest part of the screw head 210 and the screw head 210 is at its maximum point within the body 820, the screw head 210 may move downward slightly to be seated within the receiver 800. As the screw head 210 moves downward, it may press downward on the retainer ring 840 such that the retainer ring 840 is disposed between the upper 845 and lower 844 portions of the retainer ring recess 843. In some embodiments, the screw head 210 may be inserted into the opening 806 along the first axis 803 and at an angle with respect to the second axis 805. In some cases, the screw head 210 may be inserted into the opening 806 at an angle to both the first 803 and second 805 axes. In some embodiments, the screw head 210 may be inserted along the second axis 805 and at an angle with respect to the first axis 803.

As the screw head 210 is pressed upward into the receiver 800, the screw head 210 may contact and deform the coil 880 such that the coil 880 exerts a frictional force on the screw head 210. Once the screw head 210 is seated within the receiver 800, the screw head 210 may continue to contact and deform coil 880 such that the coil 880 may continue to exert a frictional force on the screw head 210.

In some embodiments, the pressure cap 860 may be aligned with the second axis 805 of the top 804 of the opening 806 and the retainer ring 840 and coil 880 may be aligned with the first axis 803 of the bottom 802 of the opening 806. However, the retainer ring 840, pressure cap 860, and coil 880 may be at any appropriate angle with respect to the first 803 and second 805 axes.

It will be understood that one or more embodiments described above may be modified in one or more ways without departing from the scope of the present disclosure. In some embodiments, a body may include fewer or more engagement features than the two engagement features shown above. In some embodiments, a receiver may not allow for modular assembly. For example, a receiver may not include the retainer ring illustrated above. In this regard, an implantable assembly may be configured such that a bottom surface of the screw head directly contacts a seating surface of the body. In some embodiments, the pressure cap may have a pin that fits into a slot of the body instead of the body having a pin that fits into the slot of the pressure cap as shown above.

Aspects, components, and features described above may be used in a variety of skeletal stabilization and/or fixation systems. For example, although the coil described above is shown in low-profile, singular receivers, the present disclosure contemplates other types of receivers and spinal implant devices. For example, the coil may be incorporated into reduction screw receiver bodies, sliding double bodies, closed receiver bodies, and/or any other suitable type of spinal implant or receiver body. Further, although embodiments of the present disclosure may be described as spinal implants or spinal fixation devices, it will be understood that the devices described above may be used for a variety of skeletal stabilization and/or fixation procedures.

Persons of ordinary skill in the art will appreciate that the implementations encompassed by the present disclosure are not limited to the particular exemplary implementations described above. In that regard, although illustrative implementations have been shown and described, a wide range of modification, change, combination, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. An implantable receiver configured to couple to a head of a bone fastener, the implantable receiver comprising:
a body comprising a top, a bottom, and an opening extending from the top to the bottom;
a pressure cap disposed within the opening of the body;
a retainer ring disposed in a bottom portion of the body within the opening, wherein the retainer ring is expandable around a head of the bone fastener; and,
a compressible coil disposed around the opening of the body above the retainer ring, wherein the compressible coil is configured to contact the head of the bone fastener and deform to provide a frictional engagement between the head of the bone fastener and the body.

2. The implantable receiver of claim 1, wherein the compressible coil is toroidally shaped.

3. The implantable receiver of claim 1, wherein the compressible coil has a cross-sectional surface that is circularly shaped.

4. The implantable receiver of claim 1, wherein the compressible coil has a cross-sectional surface that is elliptically shaped.

5. The implantable receiver of claim 4, wherein the cross-sectional surface of the compressible coil is angled outward.

6. The implantable receiver of claim 4, wherein the cross-sectional surface of the compressible coil is angled inward.

7. The implantable receiver of claim 1, wherein the compressible coil comprises a compressible polymer.

8. The implantable receiver of claim 1, wherein the compressible coil is a coiled coil.

9. The implantable receiver of claim 8, wherein the compressible coil comprises one or more of stainless steel, titanium, an alloy comprising cobalt and chromium, poly carbonate urethane (PCU), or polyether ether ketone (PEEK).

10. The implantable receiver of claim 1, wherein the compressible coil is disposed in a coil recess.

11. The implantable receiver of claim 1, wherein the body further comprises a pin that is receivable within a slot on the pressure cap.

12. A fastener assembly comprising:
a receiver comprising:
a body comprising an opening;
a pressure cap disposed within the opening of the body;
a retainer ring disposed within the opening proximate to a bottom of the opening; and,
a compressible coil disposed in the opening above the retainer ring and below the pressure cap; and,
a bone fastener comprising a head insertable through the bottom of the opening;
wherein the retainer ring is expandable around the head; and,
wherein the compressible coil is configured to deform to apply frictional force to the head.

13. The fastener assembly of claim 12, wherein the compressible coil is toroidally shaped.

14. The fastener assembly of claim 12, wherein the compressible coil has a cross-sectional surface that is circularly shaped.

15. The fastener assembly of claim 12, wherein the compressible coil has a cross-sectional surface that is elliptically shaped.

16. The fastener assembly of claim 12, wherein the compressible coil is a coiled coil.

17. The fastener assembly of claim 12, wherein the compressible coil is disposed in a coil recess.

18. A method of assembling an implantable device, the method comprising:
providing a body comprising a top, bottom, and an opening extending from the top to the bottom;
positioning a retainer ring in a recess of the opening proximate to the bottom of the body;
positioning a compressible coil in a recess of the opening above the retainer ring;
coupling a pressure cap within the opening above the compressible coil; and,
inserting a bone fastener into the bottom of the opening;
wherein the retainer ring expands around a head of the bone fastener; and,
wherein the compressible coil deforms to apply a friction force to the head.

19. The method of claim 18, wherein the compressible coil is toroidally shaped.

20. The method of claim 18, wherein the step of coupling a pressure cap includes affixing a pin to the body such that an end of the pin is received in a slot of the pressure cap.
